SEQUENCE LISTING (64 sequences)

SEQ ID NO: 1 - DNA, 26 bp, artificial sequence
  sequencing read
atttgtaaccgacttatggagcgaag

SEQ ID NO: 2 - DNA, 26 bp, artificial sequence
  Sequencing Read
atttgtaaccgactaatggagcgaag

SEQ ID NO: 3 - DNA, 66 bp, artificial sequence
  Sequencing Read
ctcgtacgaatccgttagcataccctggagtgaggtcgatgagagctcaggagatgttgactcagg

SEQ ID NO: 4 - DNA, 66 bp, artificial sequence
  Sequencing Read
ctcgtacgaatccgtttcggaaccatcgatcaagctatcaacaaaaacccataacaaactaataac

SEQ ID NO: 5 - DNA, 66 bp, artificial sequence
  Sequencing Read
ctcgtacgaatccgttaaagcgttgcgcgtaggaacatacatgaaagccgatagggaacggaatag

SEQ ID NO: 6 - DNA, 66 bp, artificial sequence
  Sequencing Read
ctcgtacgaatccgtttcatgaggtcgggaaacagagacgtgtccggcaaaactacaaaaaccaaa

SEQ ID NO: 7 - DNA, 66 bp, artificial sequence
  Sequencing Read
ctcgtacgaatccgttgttccctgtgagtccgagatcgcaatatggaggaggggctgtggtctggt

SEQ ID NO: 8 - DNA, 66 bp, artificial sequence
  Sequencing Read
ctcgtacgaatccgttggtgtcgcaggtcatctggtgcctatcataattctcctatggcctcgtag

SEQ ID NO: 9 - DNA, 66 bp, artificial sequence
  Sequencing Read
ctcgtacgaatccgttcatcgggggagtgatatccgatggaatggggtaaatcgcgaagctaagga

SEQ ID NO: 10 - DNA, 66 bp, artificial sequence
  Sequencing Read
ctcgtacgaatccgttccctacgacaacacagaaggctgcgtataaaaataaacaaaaccaataaa

SEQ ID NO: 11 - DNA, 66 bp, artificial sequence
  Sequencing Read
ctcgtacgaatccgttattaagccaaaagctagcgaggatagaggtcctaagcgctcacgtcggca

SEQ ID NO: 12 - DNA, 66 bp, artificial sequence
  Sequencing Read
ctcgtacgaatccgttccctgcgttctgtgcgacatctaagtatcacgaaaaaaaccaccaaacca

SEQ ID NO: 13 - DNA, 66 bp, artificial sequence
  Sequencing Read
ctcgtacgaatccgtttcatcgacctcactccagggtatgctgacctcactcgcgacaaaagaaaa

SEQ ID NO: 14 - DNA, 66 bp, artificial sequence
  Sequencing Read
ctcgtacgaatccgttgttgatagcttgatcgatggttccgaacaaaaaaaaaaaaactaaaaata

SEQ ID NO: 15 - DNA, 66 bp, artificial sequence
  Sequencing Read
ctcgtacgaatccgttatgtatgttcctacgcgcaacgctttgaggggtggtgcggattggaagta

SEQ ID NO: 16 - DNA, 66 bp, artificial sequence
  Sequencing Read
ctcgtacgaatccgttcacgtctctgtttcccgacctcatgacaacccaagaaacaaaaaaaaaca

SEQ ID NO: 17 - DNA, 66 bp, artificial sequence
  Sequencing Read
ctcgtacgaatccgttattgcgatctcggactcacagggaacgggcgcggtggcgggggcgggggc

SEQ ID NO: 18 - DNA, 66 bp, artificial sequence
  Sequencing Read
ctcgtacgaatccgttataggcaccagatgacctgcgacaccaatcaacgtaaaaaaccaacctag

SEQ ID NO: 19 - DNA, 66 bp, artificial sequence
  Sequencing Read
ctcgtacgaatccgttttccatcggatatcactcccccgatggtctgtcacaggagtaaggcatgc

SEQ ID NO: 20 - DNA, 66 bp, artificial sequence
  Sequencing Read
ctcgtacgaatccgttacgcagccttctgtgttgtcgtaggggggcgtaagcggctcttgccaata

SEQ ID NO: 21 - DNA, 66 bp, artificial sequence
  Sequencing Read
ctcgtacgaatccgttctatcctcgctagcttttggcttaataaaaaaaaacaaacaaaaaaaaaa

SEQ ID NO: 22 - DNA, 66 bp, artificial sequence
  Sequencing Read
ctcgtacgaatccgttacttagatgtcgcacagaacgcaggggggctcgggagccataggtcgtcc

SEQ ID NO: 23 - DNA, 43 bp, artificial sequence
  Sequencing Read
ctcgtacgaatccgttagcataccctggagtgaggtcgatgag

SEQ ID NO: 24 - DNA, 43 bp, artificial sequence
  Sequencing Read
ctcgtacgaatccgtttcggaaccatcgatcaagctatcaaca

SEQ ID NO: 25 - DNA, 43 bp, artificial sequence
  Sequencing Read
ctcgtacgaatccgttaaagcgttgcgcgtaggaacatacatg

SEQ ID NO: 26 - DNA, 43 bp, artificial sequence
  Sequencing Read
ctcgtacgaatccgtttcatgaggtcgggaaacagagacgtgt

SEQ ID NO: 27 - DNA, 43 bp, artificial sequence
  Sequencing Read
ctcgtacgaatccgttgttccctgtgagtccgagatcgcaata

SEQ ID NO: 28 - DNA, 43 bp, artificial sequence
  Sequencing Read
ctcgtacgaatccgttggtgtcgcaggtcatctggtgcctatc

SEQ ID NO: 29 - DNA, 43 bp, artificial sequence
  Sequencing Read
ctcgtacgaatccgttcatcgggggagtgatatccgatggaat

SEQ ID NO: 30 - DNA, 43 bp, artificial sequence
  Sequencing Read
ctcgtacgaatccgttccctacgacaacacagaaggctgcgta

SEQ ID NO: 31 - DNA, 42 bp, artificial sequence
  Sequencing Read
ctcgtacgaatccgttattaagccaaaagctagcgaggatag

SEQ ID NO: 32 - DNA, 43 bp, artificial sequence
  Sequencing Read
ctcgtacgaatccgttccctgcgttctgtgcgacatctaagta

SEQ ID NO: 33 - DNA, 42 bp, artificial sequence
  Sequencing Read
ctcgtacgaatccgtttcatcgacctcactccagggtatgct

SEQ ID NO: 34 - DNA, 43 bp, artificial sequence
  Sequencing Read
ctcgtacgaatccgttgttgatagcttgatcgatggttccgaa

SEQ ID NO: 35 - DNA, 43 bp, artificial sequence
  Sequencing Read
ctcgtacgaatccgttatgtatgttcctacgcgcaacgctttg

SEQ ID NO: 36 - DNA, 43 bp, artificial sequence
  Sequencing Read
ctcgtacgaatccgttcacgtctctgtttcccgacctcatgac

SEQ ID NO: 37 - DNA, 43 bp, artificial sequence
  Sequencing Read
ctcgtacgaatccgttattgcgatctcggactcacagggaacg

SEQ ID NO: 38 - DNA, 43 bp, artificial sequence
  Sequencing Read
ctcgtacgaatccgttataggcaccagatgacctgcgacacca

SEQ ID NO: 39 - DNA, 42 bp, artificial sequence
  Sequencing Read
ctcgtacgaatccgttttccatcggatatcactcccccgatg

SEQ ID NO: 40 - DNA, 43 bp, artificial sequence
  Sequencing Read
ctcgtacgaatccgttacgcagccttctgtgttgtcgtagggg

SEQ ID NO: 41 - DNA, 43 bp, artificial sequence
  Sequencing Read
ctcgtacgaatccgttctatcctcgctagcttttggcttaata

SEQ ID NO: 42 - DNA, 43 bp, artificial sequence
  Sequencing Read
ctcgtacgaatccgttacttagatgtcgcacagaacgcagggg

SEQ ID NO: 43 - DNA, 42 bp, artificial sequence
  Sequencing Read
ctcgtacgaatccgttagcataccctggagtgaggtcgatga

SEQ ID NO: 44 - DNA, 42 bp, artificial sequence
  Sequencing Read
ctcgtacgaatccgtttcggaaccatcgatcaagctatcaac

SEQ ID NO: 45 - DNA, 42 bp, artificial sequence
  Sequencing Read
ctcgtacgaatccgttaaagcgttgcgcgtaggaacatacat

SEQ ID NO: 46 - DNA, 42 bp, artificial sequence
  Sequencing Read
ctcgtacgaatccgtttcatgaggtcgggaaacagagacgtg

SEQ ID NO: 47 - DNA, 42 bp, artificial sequence
  Sequencing Read
ctcgtacgaatccgttgttccctgtgagtccgagatcgcaat

SEQ ID NO: 48 - DNA, 42 bp, artificial sequence
  Sequencing Read
ctcgtacgaatccgttggtgtcgcaggtcatctggtgcctat

SEQ ID NO: 49 - DNA, 42 bp, artificial sequence
  Sequencing Read
ctcgtacgaatccgttcatcgggggagtgatatccgatggaa

SEQ ID NO: 50 - DNA, 42 bp, artificial sequence
  Sequencing Read
ctcgtacgaatccgttccctacgacaacacagaaggctgcgt

SEQ ID NO: 51 - DNA, 42 bp, artificial sequence; identical to SEQ ID NO: 31
  Sequencing Read

SEQ ID NO: 52 - DNA, 42 bp, artificial sequence
  Sequencing Read
ctcgtacgaatccgttccctgcgttctgtgcgacatctaagt

SEQ ID NO: 53 - DNA, 30 bp, artificial sequence
  Sequencing Read
ctcgtacgaatccgttagcataccctggag

SEQ ID NO: 54 - DNA, 12 bp, artificial sequence
  Sequencing Read
tgaggtcgatga

SEQ ID NO: 55 - DNA, 30 bp, artificial sequence
  Sequencing Read
ctcgtacgaatccgttaaagcgttgcgcgt

SEQ ID NO: 56 - DNA, 12 bp, artificial sequence
  Sequencing Read
aggaacatacat

SEQ ID NO: 57 - DNA, 30 bp, artificial sequence
  Sequencing Read
ctcgtacgaatccgtttcatgaggtcggga

SEQ ID NO: 58 - DNA, 12 bp, artificial sequence
  Sequencing Read
aacagagacgtg

SEQ ID NO: 59 - DNA, 30 bp, artificial sequence
  Sequencing Read
ctcgtacgaatccgttgttccctgtgagtc

SEQ ID NO: 60 - DNA, 12 bp, artificial sequence
  Sequencing Read
cgagatcgcaat

SEQ ID NO: 61 - DNA, 30 bp, artificial sequence
  Sequencing Read
ctcgtacgaatccgttcatcgggggagtga

SEQ ID NO: 62 - DNA, 12 bp, artificial sequence
  Sequencing Read
tatccgatggaa

SEQ ID NO: 63 - DNA, 30 bp, artificial sequence
  Sequencing Read
ctcgtacgaatccgttccctacgacaacac

SEQ ID NO: 64 - DNA, 12 bp, artificial sequence
  Sequencing Read
agaaggctgcgt

(19) United States
(12) Patent Application Publication
(10) Pub. No.: US 2024/0021270 A1
(43) Pub. Date: Jan. 18, 2024

(54) METHODS, SYSTEMS AND DEVICES FOR PROCESSING SEQUENCE DATA

(71) Applicant: NanoString Technologies, Inc., Seattle, WA (US)

(72) Inventor: Peter ASKOVICH, Seattle, WA (US)

(73) Assignee: NanoString Technologies, Inc., Seattle, WA (US)

(21) Appl. No.: 18/030,889

(22) PCT Filed: Oct. 8, 2021
(86) PCT No.: PCT/US2021/054215

§ 371 Date: Apr. 7, 2023

Related U.S. Application Data
(60) Provisional application No. 63/089,432, filed on Oct. 8, 2020.

(51) Int. Cl. G16B 30/00; G16B 50/00

(52) U.S. Cl. CPC G16B 30/00; G16B 50/00

(57) ABSTRACT

Embodiments of the present disclosure are directed to systems, apparatuses, devices and methods for processing sequencing data for determining the identity of DNA fragments from a plurality of reads contained in a sequencing data file.

### RELATED APPLICATIONS

The present disclosure claims benefit of and priority to U.S. provisional patent application No. 63/089,432 filed Oct. 8, 2020, the entire disclosure of which is incorporated herein by reference in its entirety.

### FIELD OF THE DISCLOSURE

Embodiments of the present disclosure are directed to, inter alia, systems, apparatuses, and methods for determining sequences, and more particularly, determining sequences of genetic fragments, including, for example, processing sequencing reads to remove adaptor data.

### INCORPORATION BY REFERENCE OF SEQUENCE LISTING

The instant application contains a Sequence Listing which has been submitted in ASCII format via EFS-Web and is hereby incorporated by reference in its entirety. Said ASCII copy, created on Oct. 8, 2021, is named “NATE-050_001WO_SeqList ST25.txt” and is about 14 kilobytes in size.

### BACKGROUND

Processing of genetic data is a time consuming and arduous task. Sequencing reads result in voluminous amounts of data that must be processed to generate resulting data for determining a desired genetic sequence (e.g., sequences of genetic fragments). Accordingly, processes for speeding up processing of such data are desirable to provide faster results.

### SUMMARY

Embodiments disclosed herein enable an increase (and in some embodiments, a substantial increase) in processing speed of processing genetic data, and an improvement in the specificity of results thereof.

Accordingly, in some embodiments, a sequencing data processing method for aiding in the determination of the identity of DNA (in some embodiments, fragments of DNA) from a plurality of sequencing reads contained in a sequencing data file is provided. The method includes, performing a plurality of adapter trimming passes. The adapter trimming passes includes at least a first trimming pass, for each sequencing read, starting at a base pair (“bp”) that is 1 base greater than the known insert length (in some embodiments, at least 1 base greater, and in some embodiments, a predetermined number of bases greater), where adapter bps can be removed from the sequence where a first predetermined number of bps of the adapter is used so as to find a match in the sequence considering a limited plurality of possible overlaps, and after the first trimming pass, if the read is greater than a predetermined number of bps, performing a limited number of second trimming passes, at any place along the read, each including matching one or more adapters at the first predetermined number of bp of the adapter plus or minus a predetermined number of additional bps from a prior trimming pass. The limited number of trimming passes result each single-ended read being ultimately trimmed to a single-ended specific number of bps, and each paired-end read being ultimately trimmed to a paired-end specific number of bps. In some embodiments, the method may also include optionally re-labeling the/an insert bps using information from one or more trimming passes.

In such embodiments, one and/or another (and in some embodiments, a plurality of, a majority of, substantially all of, and in some embodiments, all of) of the following additional features, functions, functionality, steps, and/or clarifications, yielding yet further embodiments of the present disclosure:
the first trimming pass can be started at a specific bp (in some embodiments, bp 27);
the first trimming pass is only performed if the/a read can be at least 36 bps in length (in some embodiments, at least a predetermined length of bps or range of lengths of bps);
with the first trimming pass, the first predetermined number of bps of the adapter comprise 10 bps (in some embodiments, a predetermined number of bps);
the predetermined number of additional bps comprises between 1 and 2 bps (in some embodiments, a predetermined number of additional bps);
a plurality of sequencing reads from one or more sequencing data files (“SDF”);
   the plurality of sequencing reads comprise a plurality of single-ended reads and a plurality of paired-end reads,
   each single-ended read comprises a single SDF (“R1”), and each paired-end read comprises two SDFs (“R1”, “R2”),
   for a paired-end read, a first R1 of the two SDFs comprising a forward read of the paired-ended read, and a second R2 of the two SDFs comprising a reverse read of the paired-ended read;
   each SDF comprising a predetermined number of lines (in some embodiments, a plurality of, in some embodiments, at least 4 lines of information, in some embodiments, 4 lines of information), a second line thereof comprising sequencing data, and a fourth line thereof comprising quality scores for the sequencing data;
   the sequencing data of each read comprising insert data associated with base pairs (“bps”) of an insert (i.e., a DNA fragment), and second adapter data associated with bps of an associated adapter on an end of the insert; and/or
   for a paired-end, the sequence line of R1 can be from bp 1 to a last bp, and the sequence line of R2 can be from the last bp to bp 1;
performing at least one additional processing step on the plurality of sequencing reads selected from the group consisting of: stitching, extracting, first matching, deduplication, and second matching;
performing a step of stitching comprising one or more of (and preferably all of):
   for each paired end read, overlapping a first sequencing read (R1) of the paired-end read with a second sequencing read (R2) of the paired-end read and comparing the overlapped portions,
   upon the reads not matching selecting one of R1 and R2 having a higher quality score, or should the quality scores be equal:
      calculating at least one regional score for R1 and R2 progressively until one of R1 and R2 has a higher quality score, wherein calculating comprises adding quality score values for the non-matching bp, one bp to the left of the non-matching bp, and one bp to the right of each of R1 and R2, selecting the read having the higher total quality score, and
      trimming the selected read to a predetermined number of bp (e.g., 26 bp) using numbering from R1.
performing a step of extracting comprising splitting each read into a unique molecular identifier (“UMI”), and barcode;
performing a step of first matching comprising matching each read against a library (e.g., hash table) of expected bar codes with a given error rate, such that:
   if a barcode from a read is shorted, a last bp will be accorded as an “N”, so a remaining predetermined number of bps match exactly to an identifier in the library,
   if an exact match for bar code is specified, the predetermined number of bps match of a read is not performed; and
   if a match is not found, the read is saved in memory; and
performing a step of second matching comprising, for each barcode not matched via first matching (“NMBC”), matching the UMI of the NMBC amongst UMIs of previously matched barcodes via first matching, wherein if a UMI is found, the NMBC is compared to the barcode of the found UMI to confirm a match, allowing a plurality of mis-matched bps.

In some embodiments, a sequencing data processing method for aiding in the determination of the identity of DNA fragments from a plurality of reads contained in a sequencing data file is provided and comprises, for each paired end read, overlapping a first sequencing read (R1) of a paired-end read with a second sequencing read (R2) of a paired-end read and comparing the overlapped portions. Upon the reads not matching selecting one of R1 and R2 having a higher quality score, or should the quality scores be equal, calculating at least one regional score for R1 and R2 progressively until one of R1 and R2 has a higher quality score, where calculating comprises adding quality score values for the non-matching bp, one bp to the left of the non-matching bp, and one bp to the right of each of R1 and R2, selecting the read having the higher total quality score, and trimming the selected read to a predetermined number of bp (e.g., 26 bp) using numbering from R1.

In such embodiments, one and/or another (and in some embodiments, a plurality of, a majority of, substantially all of, and in some embodiments, all of) of the following additional features, functions, functionality, steps, and/or clarifications, yielding yet further embodiments of the present disclosure:
performing at least one additional processing step on the plurality of sequencing reads selected from the group consisting of: adapter trimming, extracting, first matching, deduplication, and second matching;
performing adapter trimming comprising a first trimming pass, for each sequencing read, starting at a bp that can be 1 base greater than the known insert length, comprising removing adapter bps from the sequence, which comprises using a first predetermined number of bps of the adapter so as to find a match in the sequence considering a limited plurality of possible overlaps;
optionally, after the first trimming pass, if the read is greater than a predetermined number of bps, performing a limited number of second trimming passes, at any place along the read, each comprising matching one or more adapters at the first predetermined number of bp of the adapter plus or minus a predetermined number of additional bps from a prior trimming pass, the limited number of trimming passes result in each single-ended read can be ultimately trimmed to a single-ended specific number of bps, and each paired-end read can be ultimately trimmed to a paired-end specific number of bps, and optionally re-labeling the an insert bps using information from one or more trimming passes;
the first trimming pass can be started at bp 27 (in some embodiments, a predetermined bp);
the first trimming pass is only performed if a read is at least 36 bps in length (in some embodiments, a predetermined length of bps);
with the first trimming pass, the first predetermined number of bps of the adapter comprises 10 bps (in some embodiments, a predetermined number of bps);
the predetermined number of additional bps comprises between 1 and 2 bps (in some embodiments, a predetermined range of bps);
reading a plurality of sequencing reads from one or more sequencing data files (“SDF”);
   the plurality of sequencing reads comprise a plurality of single-ended reads and a plurality of paired-end reads,
   each single-ended read comprises a single SDF (“R1”), and each paired-end read comprises two SDFs (“R1”, “R2”),
   for a paired-end read, a first R1 of the two SDFs comprising a forward read of the paired-ended read, and a second R2 of the two SDFs comprising a reverse read of the paired-ended read;
   each SDF comprising a predetermined number of lines (in some embodiments, a plurality of, in some embodiments, at least 4 lines of information, in some embodiments, 4 lines of information), a second line thereof comprising sequencing data, and a fourth line thereof comprising quality scores for the sequencing data;
   the sequencing data of each read comprising insert data associated with base pairs (“bps”) of an insert (i.e., a DNA fragment), and second adapter data associated with bps of an associated adapter on an end of the insert; and/or
   for a paired-end, the sequence line of R1 can be from base pair (“bp”) 1 to a last bp, and the sequence line of R2 can be from the last bp to bp 1;
extracting comprises splitting each read into a unique molecular identifier (“UMI”), and barcode;
performing first matching comprising matching each read against a library (e.g., hash table) of expected bar codes with a given error rate;
   if a barcode from a read is shorted, a last bp will be accorded as an “N”, so a remaining predetermined number of bps match exactly to an identifier in the library,
   if an exact match for bar code is specified, the predetermined number of bps match of a read is not performed; and
   if a match is not found, the read is saved in memory; and
performing second matching comprising for each barcode not matched via first matching (“NMBC”), matching the UMI of the NMBC amongst UMIs of previously matched barcodes via first matching, such that if a UMI is found, the NMBC is compared to the barcode of the found UMI to confirm a match, allowing a plurality of mis-matched bps,

In some embodiments, a sequencing data processing method for determining the identity of DNA fragments from a plurality of reads contained in a sequencing data file is provided and includes reading a plurality of sequencing reads from one or more sequencing data files (“SDF”). The plurality of sequencing reads comprise a plurality of single-ended reads and a plurality of paired-end reads, and each single-ended read comprises a single SDF (“R1”), and each paired-end read comprises two SDFs (“R1”, “R2”). For a paired-end read, a first R1 of the two SDFs comprising a forward read of the paired-ended read, and a second R2 of the two SDFs comprising a reverse read of the paired-ended read. Each SDF comprising 4 lines of information, a second line thereof comprising sequencing data, and a fourth line thereof comprising quality scores for the sequencing data. The sequencing data of each read comprising insert data associated with base pairs (“bps”) of an insert (i.e., a DNA fragment), and second adapter data associated with bps of an associated adapter on an end of the insert, and for a paired-end, the sequence line of R1 can be from base pair (“bp”) 1 to a last bp, and the sequence line of R2 can be from the last bp to bp 1.

The method further includes performing a plurality of processing steps on the plurality of sequencing reads, wherein the plurality of processing steps can be selected from the group consisting of: trimming, stitching, extracting, first matching, deduplication, and second matching.

In some embodiments, trimming comprises performing a plurality of adapter trimming passes, where the adapter trimming passes comprise a first trimming pass, starting at a bp that can be 1 base greater than the known insert length, and comprising removing adapter bps from the sequence, which comprises using a first predetermined number of bps of the adapter so as to find a match in the sequence considering a limited plurality of possible overlaps. Trimming also includes, after the first trimming pass, if the read is greater than a predetermined number of bps, performing a limited number of second trimming passes, at any place along the read, each comprising matching one or more adapters at the first predetermined number of bp of the adapter plus or minus a predetermined number of additional bps from a prior trimming pass.

In some embodiments, the limited number of trimming passes result each single-ended read being ultimately trimmed to a single-ended specific number of bps, and each paired-end read being ultimately trimmed to a paired-end specific number of bps. Optionally, insert bps can be re-labeled using information from one or more trimming passes.

In some embodiments, stitching comprises overlapping R1 of a paired-end read with R2 of the paired-end read and comparing the overlapped portions, such that, upon the reads not matching selecting one of R1 and R2 having a higher quality score. However, in some embodiments, should the quality scores be equal, at least one regional score for R1 and R2 can be calculated progressively until one of R1 and R2 has a higher quality score. In some embodiments, calculating comprises adding quality score values for the non-matching bp, one bp to the left of the non-matching bp, and one bp to the right of each of R1 and R2, selecting the read having the higher total quality score. Thereafter, the selected read can be trimmed to 26 bp using numbering from R1.

In some embodiments, the method further includes extracting, which comprises splitting each read into a unique molecular identifier (“UMI”), and barcode.

In some embodiments, the method further includes first matching which comprises matching each read against a library (e.g., hash table) of expected bar codes with a given error rate. If a barcode from a read is shorted, a last bp will be accorded as an “N”, so a remaining predetermined number of bps match exactly to an identifier in the library. If an exact match for bar code is specified, the predetermined number of bps match of a read is not performed, and if a match is not found, the read is saved in memory.

In some embodiments, the method also includes de-duplicating the plurality of reads.

In some embodiments, the method also includes second matching, which comprises, for each barcode not matched via first matching (“NMBC”), matching the UMI of the NMBC amongst UMIs of previously matched barcodes via first matching. If a UMI is found, the NMBC can be compared to the barcode of the found UMI to confirm a match, allowing a plurality of mis-matched bps.

In such embodiments, one and/or another (and in some embodiments, a plurality of, a majority of, substantially all of, and in some embodiments, all of) of the following additional features, functions, functionality, steps, and/or clarifications, yielding yet further embodiments of the present disclosure:
the first trimming pass can be started at bp 27 (in some embodiments, a predetermined bp);
the first trimming pass is only performed if the/a read is at least 36 bps in length (in some embodiments, at least a predetermined length of bps or range of lengths of bps);
with the first trimming pass, the first predetermined number of bps of the adapter comprise 10 bps (in some embodiments, a predetermined number of bps);
the predetermined number of additional bps comprises between 1 and 2 bps (in some embodiments, a predetermined number of additional bps);
during first matching, the remaining number of bps comprises 11 bps; and
during second matching, the plurality of allowed mis-matched bps comprises one or two bps (in some embodiments, a predetermined number of bps).

In some embodiments, a system and/or device is provided for performing any of the methods recited above/disclosed herein. Such a system/device can comprise at least one computer, which may be a server, a desktop, a laptop, a smartphone, a tablet, and/or the like, having operating thereon an application and/or computer instructions (which may be in the form of one or more application programs) configured to cause the system/device to perform any of the method embodiment recited above/disclosed herein.

Accordingly, the system/device, in some embodiments, include at least one processor having access to computer instructions configured to operate thereon and cause the system/device to perform any of the methods recited above/disclosed herein.

In some embodiments, a data storage device or system is provided and for storing data and/or computer instructions (which may be in the form of one or more application programs) operational on one or more processors for causing the one or more processors to perform any of the methods recited above/disclosed herein.

It should be appreciated that any and all combinations of the foregoing concepts and additional concepts disclosed herein (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

The above-noted embodiments will become even more evident by reference to the following detailed description and accompanying drawings.

### BRIEF DESCRIPTION OF THE DRAWINGS

The skilled artisan will understand that the drawings of this disclosure are primarily for illustrative purposes and are not intended to limit the scope of inventive subject matter described herein.

FIG. 1 is sequencing data read out from 10 sequencing reads (e.g., paired-end reads) from a data sequencing file (e.g., fastq), according to some embodiments; the depicted sequences correspond to SEQ ID NOs 3-22;

FIG. 2A is a result of a trimming process applied to a first read of the paired-end read of the 10 sequencing reads from FIG. 1, according to some embodiments; the depicted sequences correspond to SEQ ID NOs 23-32;

FIG. 2B is a result of a trimming process applied to a second read of the paired-end read of the 10 sequencing reads from FIG. 1, according to some embodiments; the depicted sequences correspond to SEQ ID NOs 33-42;

FIG. 3 is a result of a stitching process applied to the 10 sequencing reads from FIG. 1, according to some embodiments; the depicted sequences correspond to SEQ ID NOs 43-52; and

FIG. 4 is a result of a first matching process of the reads from FIG. 1, according to some embodiments the depicted sequences correspond to SEQ ID NOs 53-64.

FIG. 5 is an exemplary system, and components thereof, for performing sequencing data processing, according to some embodiments.

### DETAILED DESCRIPTION

Embodiments of the present disclosure are directed to methods, systems, and devices, for processing sequencing data, and in particular performing various processes to sequencing reads. According, in some embodiments, a sequencing data processing method for determining the identity of DNA fragments from a plurality of reads contained in a sequencing data file is provided.

One of the salient features of at least some of the embodiments of the present disclosure, is utilizing the known fragment/insert size of the sequencing read, which allows at least several processing steps of at least some embodiments of the sequencing data processing methods to be sped up, thus resulting in a faster processing of sequencing data over the state of the art.

Initially, a plurality of sequencing reads are read from one or more sequencing data files (“SDF”), which, for example, can be fastq files. A fastq file comprises a text-based format for storing both a biological sequence (e.g., nucleotide sequence), as well as corresponding quality scores. Accordingly, a sequence letter and an associated quality score are each encoded with a single ASCII character. Fastq files are a commonly used format for storing the output of high-throughput sequencing instruments. Examples of such sequencing instruments include the MiSeg™, NovaSeg™, NextSeq™550 and NexSeq™2K instruments from Illumina, Inc. (San Diego, California).

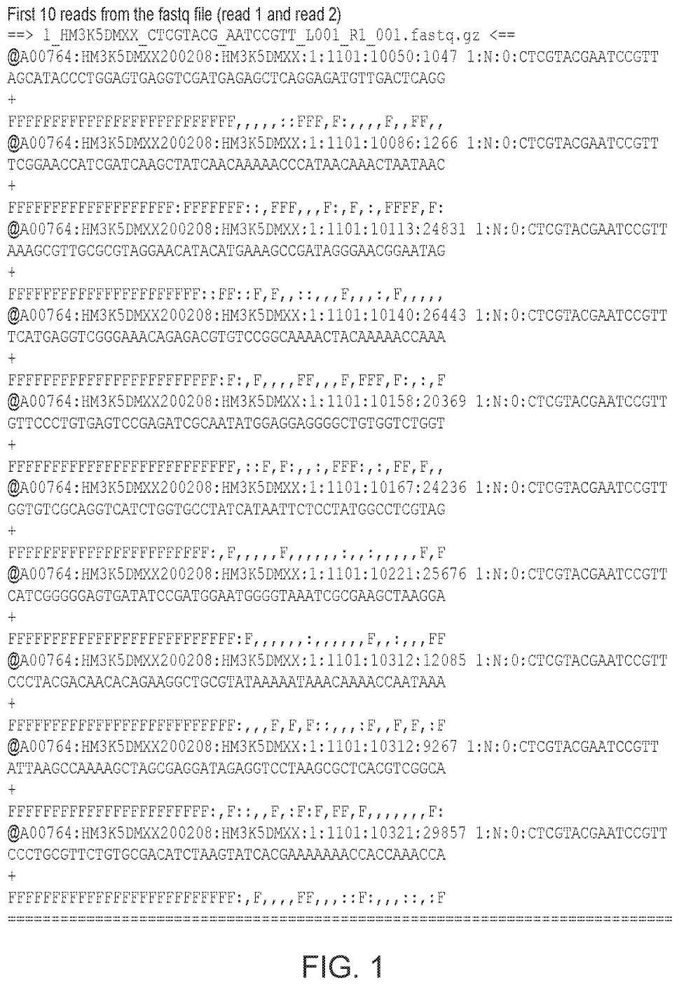

The plurality of sequencing reads comprise at least one of, and preferably, both of a plurality of single-ended reads and a plurality of paired-end reads. Each single-ended read comprises a single SDF (referred to here as “R1”), and each paired-end read comprises two SDFs (referred to respectively here as “R1”, “R2”). Accordingly, for a paired-end read, a first R1 of the two SDFs (R1 and R2) comprise a forward read of the paired-ended read, and R2 of the two SDFs comprises a reverse read of the paired-ended read. FIG. 1 is illustrative of such sequencing reads (e.g., 10, paired-end sequencing reads).

In some embodiments, each SDF is made up of four (4) lines of information, where one line (e.g., a second line) of the SDF including sequencing data, and another line (e.g., a fourth line) of the SDF is made up of associated quality scores for the sequencing data. The sequencing data/line of each read also includes insert data associated with base pairs (“bps”) of an insert (e.g., a DNA fragment), and adapter data associated with bps of an associated adapter on an end of the insert. For a paired-end, the sequence line of R1 can be from base pair (“bp”) 1 to a last bp, and the sequence line of R2 can be from the last bp to bp 1.

In some embodiments, the method further includes performing at least one processing step on at least one sequencing read, and preferably on a plurality of sequencing reads, and in some embodiments a plurality of processing steps. Such processing steps include, for example, trimming, stitching, extracting, first matching, deduplication, and second matching.

In some embodiments, trimming can be used to remove, for example, adapter information from insert information from one or more sequencing reads. Such trimming, in some embodiments, includes performing a plurality of adapter trimming passes. For example, in some embodiments, a first trimming pass can be conducted, starting at a bp that can be 1 base greater than the known insert length (in some embodiments, the first trimming pass can be initiated at a different base position greater or lesser than the known insert length, e.g., 2, 3, 4). In some embodiments, the first trimming pass can be initiated at bp 27. Additionally, in some embodiments, the first trimming pass is only performed if a read is at least a predetermined number of bps in length; for example, at least 36 bps in length.

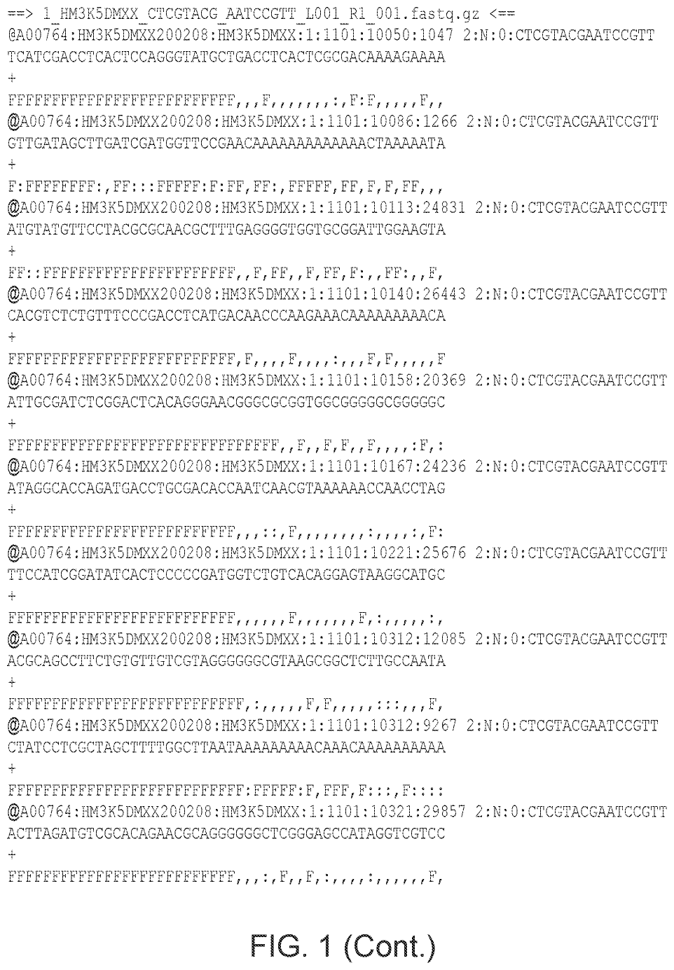

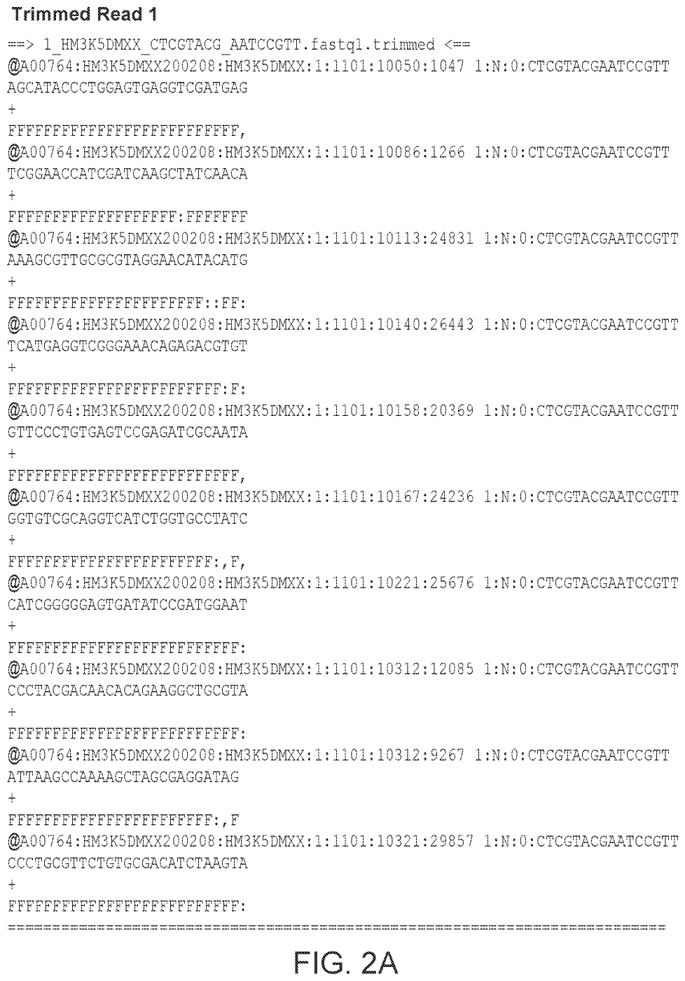

The first trimming pass, in some embodiments, removes adapter bps from the sequence read, using a first predetermined number of bps of the adapter so as to find a match in the sequence considering a limited plurality of possible overlaps. In some embodiments, the first predetermined number of bps comprise 10 bps. After the first trimming pass, in some embodiments, if the resulting read is greater than a predetermined number of bps, a limited number of second trimming passes can be performed at any place along the read. In each second trimming pass, one or more adapters can be matched at the first predetermined number of bp of the adapter plus or minus a predetermined number of additional bps from a prior trimming pass. In some embodiments, the predetermined number of additional bps comprises between 1 and 2 bps. FIGS. 2A and 2B are illustrative of the results of trimming processing of the reads of FIG. 1, according to such embodiments of the present disclosure.

In some embodiments, the limited number of trimming passes result each single-ended read being ultimately trimmed to a single-ended specific number of bps, and each paired-end read being ultimately trimmed to a paired-end specific number of bps. Optionally, insert bps can be re-labeled using information from one or more trimming passes.

Accordingly, after adapter trimming, in some embodiments, the sequencing data processing method can also include stitching of the sequencing reads. Stitching, in some embodiments, comprises overlapping R1 of a paired-end read with R2 of the paired-end read, and then comparing the overlapped portions. If the reads do not match, the stitching process includes selecting the read (of R1 and R2) having a higher quality score.

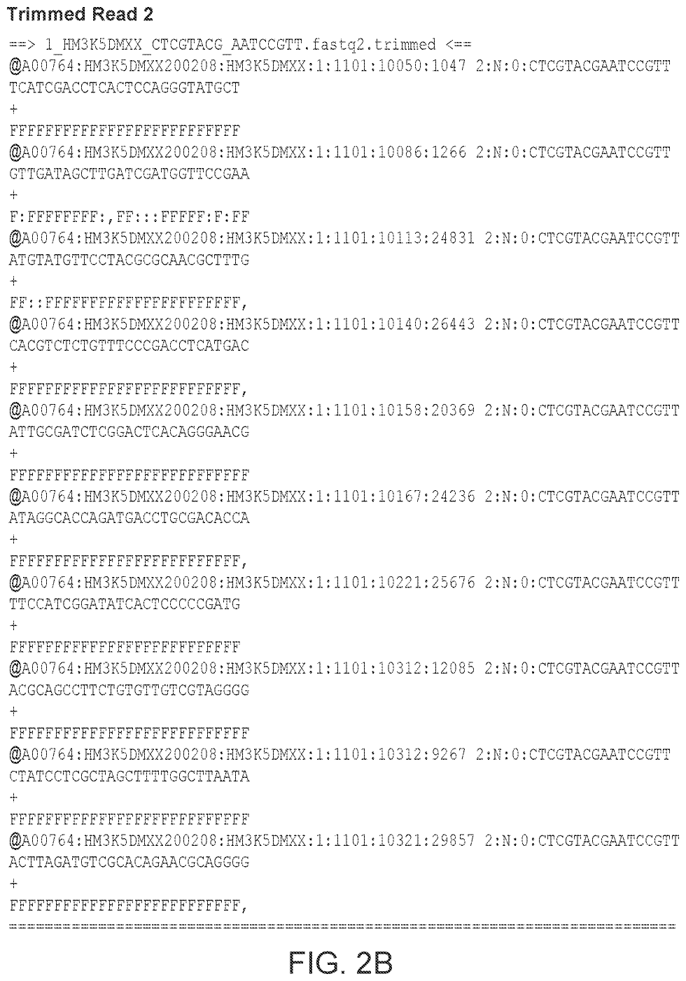

However, should the quality scores be equal, in some embodiments, the stitching process includes progressively calculating at least one regional score for R1 and R2 until one of the reads (R1 and R2) has a higher quality score than the other. Such calculating, in some embodiments, comprises adding quality score values for the non-matching bp a predetermined number of bps to the left of the non-matching bp, and to the right, of each of R1 and R2 (e.g., one bp), and then selecting the read which results in the higher total quality score. Thereafter, the selected read can be trimmed to 26 bp using numbering from R1. FIG. 3 is illustrative of the results of the stitching processing of the reads of FIG. 1.

For example, as shown below, for two (2) reads, R1 and R2, R1 is used as is, while R2 is used as a reverse complement (since being the other strand). The letters above and below sequences are the corresponding quality scores for each read. Accordingly, where F is greater than: (37 vs 25)

| | | |
|---|---|---|
| | FFFFFFFFFFFFF:FFFFFFFFFF:F | |
| R1 | ATTTGTAACCGACTTATGGAGCGAAG | (SEQ ID NO: 1) |
| R2 | ATTTGTAACCGACTAATGGAGCGAAG | (SEQ ID NO: 2) |
| | FFFFFFFFFFFFFFFFFFFFFFFFFF | |

At position 15, R1 includes bp T, and at the same location in R2, there is an A, and both bases include the same quality score (37). In order to determine which read to use, regional scores of each read are calculated by adding quality score values of one bp to the left, and one bp to the right of the bp at issue (i.e., bp 15):

R1=:FF=25+37+37=99

R2=FFF=37+37+37=111

In this example, R2 wins, as the calculated regional score is greater (111 vs. 99). Thus, the resulting final sequence is:

(SEQ ID NO: 2)
ATTTGTAACCGACTAATGGAGCGAAG

If, in the case where adding the quality scores of adjacent bps (e.g., −1 and +1) still produces the same score, in some embodiments, quality scores of other still further away bps are added (e.g., −2 and +2) until a different result is obtained between the reads. Accordingly, as stated above, the above regional scoring process can be further modified with respect to other “calculating” of other respective scoring and the like, so as to select a sequencing read.

In some embodiments, the sequencing data processing method can further include an extracting process, which comprises splitting each read into a unique molecular identifier (“UMI”), and barcode.

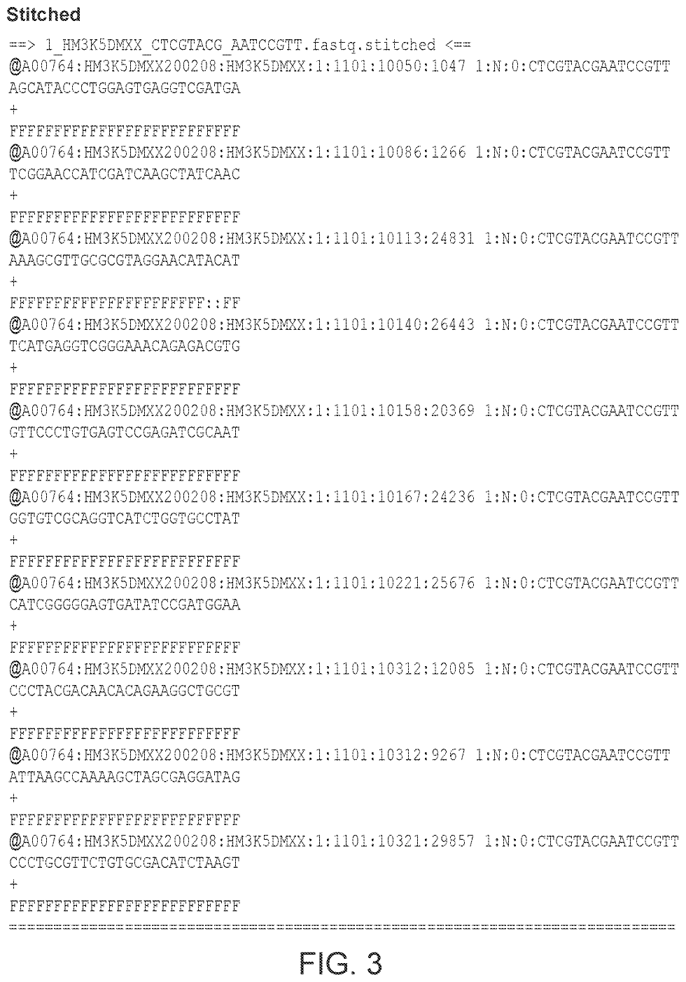

In some embodiments, the method can further include a first matching step. The first matching step comprises matching each read against a library (e.g., hash table, and/or the like) of expected bar codes with a given error rate. Accordingly, in this process, if a barcode from a read is “shorted”, such that, a last bp will be accorded as an “N”, which can be any base. In some embodiments, matching can be allowed to occur with one (1) error (i.e., mismatch). Accordingly, if the last base is missing (due to the sequence being short), “N” can be added which will not match, because it is not any of A, C, G, or T. Thereafter, an exact match can then be required from the remaining 11 bps. Thus, a remaining predetermined number of bps match exactly to an identifier in the library. In some embodiments, if an exact match for a bar code is specified, the predetermined number of bps match of a read is not performed, and/or if a match is not found, the read can be saved in memory. In some embodiments, during first matching, the remaining number of bps comprises for example, 11 bps. FIG. 4 is illustrative of such a matching process for the reads of FIG. 1, after trimming (FIGS. 2A-B).

In some embodiments, the method also includes de-duplicating the plurality of reads (see, e.g., Smith, T. S., et al., UMI-tools: Modelling sequencing errors in Unique Molecular Identifiers to improve quantification accuracy; Cold Spring Harbor Laboratory Press; Jan. 18, 2017, hereinafter incorporated by reference).

In some embodiments, the method also includes second matching. Second matching, in some embodiments, is a process that, for each barcode not matched via first matching (non-matching barcode or “NMBC”), second matching matches the UMI of the NMBC among UMIs of previously matched barcodes (which were matched via first matching). Accordingly, if a UMI is found, the NMBC can be compared to the barcode of the found UMI to confirm a match, allowing a plurality of mis-matched bps. In some embodiments, during second matching, the plurality of allowed mis-matched bps can comprise one or two bps (for example). To this end, at least some of the method and system embodiments disclosed herein can be used in conjunction with the embodiments described in US2019/0249248A1, to assemble sequences of the amplification products from the probes described therein, thereby ascertaining the identifier oligonucleotides and spatially detecting a target analyte.

### Sequencing Data Processing Systems and Software

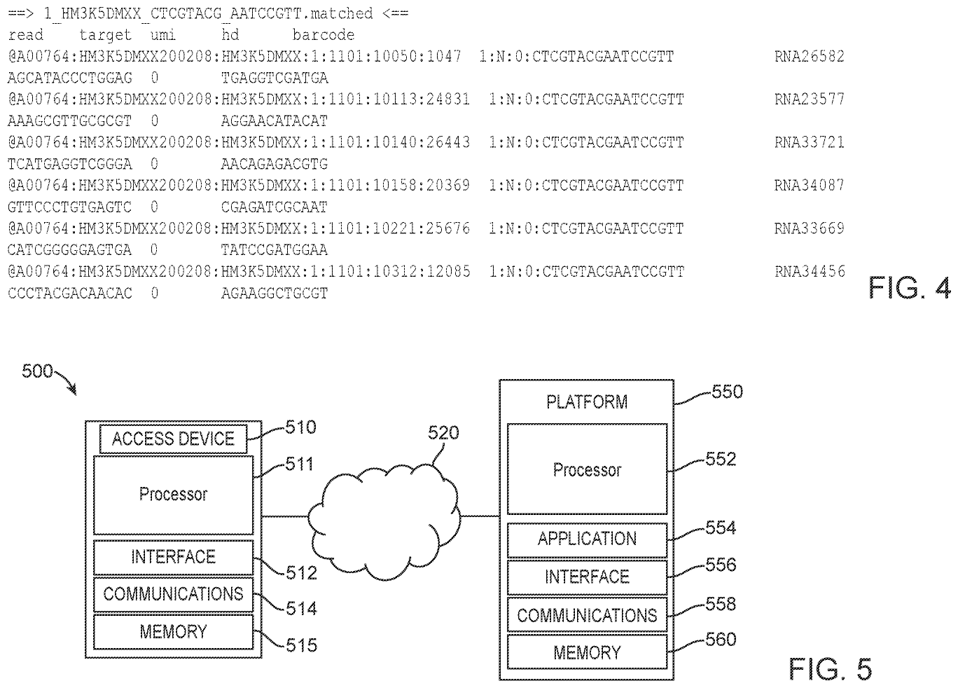

One and/or another of the above-noted process embodiments (and/or steps thereof) can be carried out on one or more computing devices/systems (and/or components thereof), an example of which can be found in FIG. 5. As shown, system 500, which can include, e.g.

access device 510, platform 550, and network 520. Such systems, devices, and platforms may include one or more processors 511, 552 (e.g., microprocessors, CPUs, GPUs, etc.), one or more computer-readable RAMs, one or more computer-readable ROMs, one or more computer readable storage media (all of the preceding can be referred to as memory 515, 560, but can be separate structure—e.g., remote data storage facilities—communicating with, and/or with components of, system 500). Other components/functionality can include device drivers, read/write drives, interfaces (e.g., 512, 556), network adapter or interface, all interconnected over a communications network(s) 520 (via e.g., 514, 558, which can be referred to as a network adapter). The network adapter communicates with the network 520; the communications network(s) may be implemented with any architecture designed for passing data and/or control information between processors (such as microprocessors, communications and network processors, etc.), system memory, peripheral devices, and any other hardware components within a system.

One or more operating systems and one or more application programs (e.g., 554), such as a sequencing data processing application according to embodiments of the disclosure, which can reside on a sequencing data platform 550, can be stored on one or more of the computer readable storage media for execution by one or more of the processors via one or more of the respective RAMs (which typically include cache memory). In some embodiments, each of the computer readable storage media may be a magnetic disk storage device of an internal hard drive, CD-ROM, DVD, memory stick, magnetic tape, magnetic disk, optical disk, a semiconductor storage device such as RAM, ROM, EPROM, flash memory or any other computer-readable medium (e.g., a tangible storage device) that can store a computer program and digital information.

The user device and/or sequencing data processing system/platform may also include a read/write (R/W) drive or interface to read from and write to one or more portable computer readable storage media (or cloud based data storage). Application programs on a viewing device and/or user device (e.g., 510) may be stored on one or more of the portable computer readable storage media, read via the respective R/W drive or interface and loaded into the respective computer readable storage media. The user device and/or the sequencing data processing system/platform may also include the network adapter or interface, such as a Transmission Control Protocol (TCP)/Internet Protocol (IP) adapter card or wireless communication adapter (such as a 4G, 5G wireless communication adapter using Orthogonal Frequency Division Multiple Access (OFDMA) technology). For example, application programs may be downloaded to a computing device from an external computer or external storage device via a network (for example, 520, the Internet, a local area network or other wide area network or wireless network) and network adapter or interface. From the network adapter or interface, the programs may be loaded onto computer readable storage media. The network may include copper wires/cables, optical fibers/cables, wireless transmission, routers, firewalls, switches, gateway computers and/or edge servers. User device and/or the sequencing data processing system/platform may also include one or more output devices or interfaces (e.g., a display screen), and one or more input devices or interfaces (e.g., keyboard, keypad, mouse or pointing device, touchpad). For example, device drivers may interface to output devices or interfaces for imaging, to input devices or interfaces for user input or user selection (e.g., via pressure or capacitive sensing), and so on. The device drivers, R/W drive or interface and network adapter or interface may include hardware and software (stored on computer readable storage media and/or ROM).

In some embodiments, the sequencing data processing system/platform (as well as the methodology thereof) can be a standalone network server or represent functionality integrated into one or more network systems. User device 510 and/or the sequencing data processing system/platform 550 can be a laptop computer, desktop computer, specialized computer server, or any other computer system known in the art. In some embodiments, the sequencing data processing system represents computer systems using clustered computers and components to act as a single pool of seamless resources when accessed through a network (e.g., 520), such as a LAN, WAN, or a combination of the two. This embodiment may be desired, particularly for data centers and for cloud computing applications. In general, user device and/or the sequencing data processing system can be any programmable electronic device or can be any combination of such devices, in accordance with embodiments of the present disclosure.

The programs described herein are identified based upon the application for which they are implemented in a specific embodiment or embodiment(s) of the present disclosure. That said, any particular program nomenclature herein is used merely for convenience, and thus the embodiments and embodiments of the present disclosure should not be limited to use solely in any specific application identified and/or implied by such nomenclature.

Embodiments of the present disclosure may be or use one or more of a device, system, method (e.g., see above), and/or computer readable medium at any possible technical detail level of integration. The computer readable medium may include a computer readable storage medium (or media) having computer readable program instructions thereon for causing a processor to carry out one or more aspects of the present disclosure. The computer readable (storage) medium can be a tangible device that can retain and store instructions for use by an instruction execution device. The computer readable medium may be, but is not limited to, for example, non-transitory storage media, including an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire, in accordance with embodiments of the present disclosure.

Computer readable program instructions described herein, as noted above, can be downloaded to respective computing/processing devices from a computer readable storage medium or to an external computer or external storage device via a network, for example, the Internet, a local area network, a wide area network and/or a wireless network. The network may comprise copper wire/cable(s), optical fiber/cable(s), wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network (e.g., 520), including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, to perform various aspects of the present disclosure.

These computer readable program instructions may be provided to a processor of a general purpose computer, special purpose computer, or other programmable data processing apparatus to produce a machine or system (e.g., see above), such that the instructions, which execute via the processor of the computer or other programmable data processing apparatus, create means for implementing the functions/acts/steps/processes specified in this disclosure (for any disclosed method embodiments). These computer readable program instructions may also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified herein, in accordance with embodiments of the present disclosure.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in herein.

Various inventive concepts disclosed herein may be embodied as one or more methods (as so noted), of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Any and all references to publications or other documents, including but not limited to, patents, patent applications, articles, webpages, books, etc., presented anywhere in the present application, are herein incorporated by reference in their entirety.

As noted elsewhere, the disclosed inventive embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, systems and apparatuses/devices which may further include any and all elements from any other disclosed methods, systems, and devices, including any and all elements corresponding to binding event determinative systems, devices and methods. In other words, elements from one or another disclosed embodiments may be interchangeable with elements from other disclosed embodiments. In addition, one or more features/elements of disclosed embodiments may be removed and still result in patentable subject matter (and thus, resulting in yet more embodiments of the subject disclosure). Also, some embodiments correspond to systems, devices and methods which specifically lack one and/or another element, structure, and/or steps (as applicable), as compared to teachings of the prior art, and therefore, represent patentable subject matter and are distinguishable therefrom (i.e., claims directed to such embodiments may contain negative limitations to note the lack of one or more features prior art teachings).

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to) for a particular embodiment(s).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A sequencing data processing method for aiding in the determination of the identity of DNA fragments from a plurality of reads contained in a sequencing data file, the method comprising
performing a plurality of adapter trimming passes, the adapter trimming passes comprising at least:
a first trimming pass, for each sequencing read, starting at a base pair (bp) that is 1 base greater than the known insert length, comprising removing adapter bps from the sequence, which comprises using a first predetermined number of bps of the adapter so as to find a match in the sequence considering a limited plurality of possible overlaps;
after the first trimming pass, if the sequencing read is greater than a predetermined number of bps, performing a limited number of second trimming passes, at any place along the sequencing read, each comprising matching one or more adapters at the first predetermined number of bp of the adapter plus or minus a predetermined number of additional bps from a prior trimming pass, wherein:
the limited number of trimming passes result each single-ended read is ultimately trimmed to a single-ended specific number of bps, and each paired-end read is ultimately trimmed to a paired-end specific number of bps; and
optionally re-labeling an insert bps using information from one or more trimming passes.

2. The method of claim 1, wherein the first trimming pass is started at bp 27.

3. The method of claim 1, wherein the first trimming pass is only performed if the sequencing read is at least 36 bps in length.

4. The method of claim 1, wherein with the first trimming pass, the first predetermined number of bps of the adapter comprises 10 bps.

5. The method of claim 1, wherein the predetermined number of additional bps comprises between 1 and 2 bps.

6. (canceled)

7. The method of claim 1, further comprising reading a plurality of sequencing reads from one or more sequencing data files (“SDF”), wherein:
the plurality of sequencing reads comprise a plurality of single-ended reads and a plurality of paired-end reads,
each single-ended read comprises a single SDF (“R1”), and each paired-end read comprises two SDFs (“R1”, “R2”),
for a paired-end read, a first R1 of the two SDFs comprising a forward read of the paired-ended read, and a second R2 of the two SDFs comprising a reverse read of the paired-ended read;
each SDF comprising 4 lines of information, a second line thereof comprising sequencing data, and a fourth line thereof comprising quality scores for the sequencing data;
the sequencing data of each read comprising insert data associated with basepairs (“bps”) of an insert (i.e., a DNA fragment), and second adapter data associated with bps of an associated adapter on an end of the insert; and/or
for a paired-end, the sequence line of R1 is from base pair (“bp”) 1 to a last bp, and the sequence line of R2 is from the last bp to bp 1.

8. (canceled)

9. The method of claim 7, further comprising performing at least one additional processing step on the plurality of sequencing reads, wherein the at least one additional processing step is selected from the group consisting of:
stitching, extracting, first matching, deduplication, and second matching.

10. The method of claim 9, wherein the stitching comprises:
for each paired end read, overlapping a first sequencing read (R1) of the paired-end read with a second sequencing read (R2) of the paired-end read and comparing the overlapped portions,
wherein upon the reads not matching, selecting one of R1 and R2 having a higher quality score, or should the quality scores be equal:
calculating at least one regional score for R1 and R2 progressively until one of R1 and R2 has a higher quality score, wherein calculating comprises adding quality score values for the non-matching bp, one bp to the left of the non-matching bp, and one bp to the right of each of R1 and R2, selecting the read having the higher total quality score, and
trimming the selected read to a predetermined number of bp (e.g., 26 bp) using numbering from R1.

11. The method of claim 9, wherein the extracting comprises splitting each read into a unique molecular identifier (“UMI”), and barcode.

12. The method of claim 9, wherein the first matching comprises matching each read against a library (e.g., hash table) of expected bar codes with a given error rate.

13. The method of claim 12, wherein, with respect to the first matching:
if a barcode from a read is shorted, a last bp will be accorded as an “N”, so a remaining predetermined number of bps match exactly to an identifier in the library,
if an exact match for bar code is specified, the predetermined number of bps match of a read is not performed; and
if a match is not found, the read is saved in memory.

14. The method of claim 9, wherein the second matching comprises, for each barcode not matched via alignment matching (“NMBC”), matching the UMI of the NMBC amongst UMIs of previously matched barcodes via alignment matching, wherein if a UMI is found, the NMBC is compared to the barcode of the found UMI to confirm a match, allowing a plurality of mis-matched bps.

15. A sequencing data processing method for aiding in the determination of the identity of DNA fragments from a plurality of sequencing reads contained in one or more sequencing data files (“SDF”), the method comprising a stitching process comprising:
for each paired end read, overlapping a first sequencing read (R1) of a paired-end read with a second sequencing read (R2) of a paired-end read and comparing the overlapped portions,
wherein upon the reads not matching selecting one of R1 and R2 having a higher quality score, or should the quality scores be equal:
calculating at least one regional score for R1 and R2 progressively until one of R1 and R2 has a higher quality score, wherein calculating comprises adding quality score values for the non-matching bp, one bp to the left of the non-matching bp, and one bp to the right of each of R1 and R2, selecting the read having the higher total quality score, and
trimming the selected read to a predetermined number of bp (e.g., 26 bp) using numbering from R1.

16. (canceled)

17. The method of claim 15, further comprising performing at least one additional processing step on the plurality of sequencing reads, the at least one additional processing step selected from the group consisting of: adapter trimming, extracting, first matching, deduplication, and second matching.

18. The method of claim 17, wherein the adapter trimming comprises at least:
a first trimming pass, for each sequencing read, starting at a bp that is 1 base greater than the known insert length, comprising removing adapter bps from the sequence, which comprises using a first predetermined number of bps of the adapter so as to find a match in the sequence considering a limited plurality of possible overlaps;
after the first trimming pass, if the read is greater than a predetermined number of bps, performing a limited number of second trimming passes, at any place along the read, each comprising matching one or more adapters at the first predetermined number of bp of the adapter plus or minus a predetermined number of additional bps from a prior trimming pass, wherein:
the limited number of trimming passes result each single-ended read is ultimately trimmed to a single-ended specific number of bps, and
each paired-end read is ultimately trimmed to a paired-end specific number of bps; and
optionally re-labeling the/an insert bps using information from one or more trimming passes.

19-23. (canceled)

24. The method of claim 15, wherein:
the plurality of sequencing reads comprise a plurality of single-ended reads and a plurality of paired-end reads,
each single-ended read comprises a single SDF (“R1”), and each paired-end read comprises two SDFs (“R1”, “R2”),
for a paired-end read, a first R1 of the two SDFs comprising a forward read of the paired-ended read, and a second R2 of the two SDFs comprising a reverse read of the paired-ended read;
each SDF comprising 4 lines of information, a second line thereof comprising sequencing data, and a fourth line thereof comprising quality scores for the sequencing data;
the sequencing data of each read comprising insert data associated with basepairs (“bps”) of an insert (i.e., a DNA fragment), and second adapter data associated with bps of an associated adapter on an end of the insert; and/or
for a paired-end, the sequence line of R1 is from basepair (“bp”) 1 to a last bp, and the sequence line of R2 is from the last bp to bp 1.

25. (canceled)

26. The method of claim 17, wherein the first matching comprises matching each read against a library (e.g., hash table) of expected bar codes with a given error rate.

27. The method of claim 26, wherein, with respect to the first matching:
if a barcode from a read is shorted, a last bp will be accorded as an “N”, so a remaining predetermined number of bps match exactly to an identifier in the library,
if an exact match for bar code is specified, the predetermined number of bps match of a read is not performed; and
if a match is not found, the read is saved in memory.

28. The method of claim 17, wherein the second matching comprises for each barcode not matched via alignment matching (“NMBC”), matching the UMI of the NMBC amongst UMIs of previously matched barcodes via alignment matching, wherein if a UMI is found, the NMBC is compared to the barcode of the found UMI to confirm a match, allowing a plurality of mis-matched bps.

29-35. (canceled)

36. A system or device comprising at least one computer processor having access to computer instructions configured to cause a server to perform the method recited in claim 1.

37-38. (canceled)
